(19) United States
(12) Patent Application Publication
(10) Pub. No.: US 2009/0048272 A1
(43) Pub. Date: Feb. 19, 2009

(54) PREPARATION OF PALIPERIDONE

(76) Inventors: Pratap Reddy Padi, Hyderabad (IN); Satyanarayana Bollikonda, Hyderabad (IN); Surya Prabhakar Akundi, Hyderabad (IN); Praveen Cherukupally, Hyderabad (IN); Sashikanth Suthrapu, Warangal (IN); Saravanan Mohanarangam, Villupuram (IN); Venkat Kiran Kandirelli, Hyderabad (IN); Malleswara Rao Chellu, Hyderabad (IN); Narasimha Rao Pagadala, Guntur (IN)

Correspondence Address: DR. REDDY''S LABORATORIES, INC., 200 SOMERSET CORPORATE BLVD, SEVENTH FLOOR, BRIDGEWATER, NJ, 08807-2862, US

(21) Appl. No.: 12/192,241

(22) Filed: Aug. 15, 2008

Related U.S. Application Data
(60) Provisional application No. 61/040,295, filed on Mar. 28, 2008.

(30) Foreign Application Priority Data
Aug. 16, 2007 (IN) 1825/CHE/2007
May 21, 2008 (IN) 1240/CHE/2008

(51) Int. Cl. A61K 31/519; C07D 239/70; A61P 25/00

(52) U.S. Cl. 514/259.41; 544/282

(57) ABSTRACT

Processes for preparing paliperidone.

### TECHNICAL FIELD

The present application relates to processes for preparing paliperidone.

### BACKGROUND

Paliperidone is a primary active metabolite of the older antipsychotic drug risperidone (paliperidone is 9-hydroxyrisperidone, i.e., risperidone having a hydroxyl group substituent). Paliperidone is a racemic mixture having a chemical name (±)-3-[2-[4-(6-fluoro-1,2-benzisoxazol-3-yl)-1-piperidinyl]ethyl]-6,7,8,9-tetrahydro-9-hydroxy-2-methyl-4H-pyrido[1,2-a]pyrimidin-4-one and is structurally represented by Formula I.

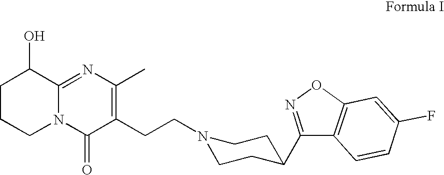

Paliperidone is a psychotropic agent belonging to the chemical class of benzisoxazole derivatives and is present in products marketed by Janssen under the brand name INVEGA™ in 3 mg, 6 mg, and 9 mg strengths. Paliperidone is indicated for the acute and maintenance treatment of schizophrenia.

U.S. Pat. No. 5,158,952 describes piperidinyl-1,2-benzisoxazoles, intermediates, and processes for their preparation. The chemical pathway exemplified for paliperidone in this patent is summarized in Scheme I.

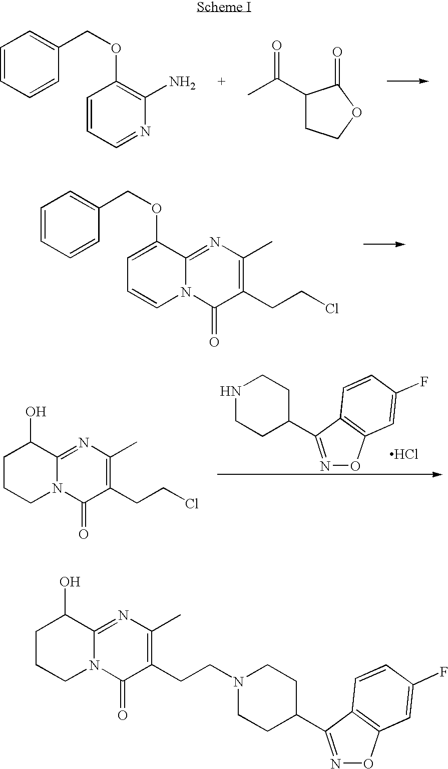

The process exemplified in this patent for the preparation of paliperidone, as well as intermediates thereof, involves isolations using column chromatography and recrystallization in suitable solvents.

There is a need to provide an economically viable process for the preparation of paliperidone and intermediates thereof which avoids column chromatography and the resulting lengthy work-ups.

### SUMMARY

The present application relates to processes for preparing paliperidone of Formula I, an embodiment comprising:

(1) reacting 3-hydroxy-2-pyridinamine of Formula VII with 3-acetyl-4,5-dihydro-2(3H)-furanone of Formula V to afford 3-(2-chloroethyl)-2-methyl-9-hydroxy-4H-pyrido[1,2-a]pyrimidin-4-one of Formula IV;

(2) reducing 3-(2-chloroethyl)-2-methyl-9-hydroxy-4H-pyrido[1,2-a]pyrimidin-4-one of Formula IV in the presence of acetic anhydride to afford 3-(2-chloroethyl)-6,7,8,9-tetrahydro-9-acetyloxy-2-methyl-4H-pyrido[1,2-a]pyrimidin-4-one of Formula VIII and optionally converting the compound VIII into its salt; and

(3) condensing 3-(2-chloroethyl)-6,7,8,9-tetrahydro-9-acetyloxy-2-methyl-4H-pyrido[1,2-a]pyrimidin-4-one of Formula VIII or its salt with 6-fluoro-3-(4-piperidinyl)-1,2-benzisoxazole of Formula II or its salt to afford (±)-3-[2-[4-(6-fluoro-1,2-benzisoxazol-3-yl)-1-piperidinyl]ethyl]-6,7,8,9-tetrahydro-9-hydroxy-2-methyl-4H-pyrido[1,2-a]pyrimidin-4-one of Formula I.

In another embodiment, a process for preparing paliperidone comprises:

(1) converting the 3-(2-chloroethyl)-6,7,8,9-tetrahydro-9-acetyloxy-2-methyl-4H-pyrido[1,2-a]pyrimidin-4-one of Formula VIII or its salt to 3-(2-chloroethyl)-6,7,8,9-tetrahydro-9-hydroxy-2-methyl-4H-pyrido[1,2-a]pyrimidin-4-one of Formula III by hydrolysis and optionally converting the compound III into its salt; and

(2) condensing 3-(2-chloroethyl)-6,7,8,9-tetrahydro-9-hydroxy-2-methyl-4H-pyrido[1,2-a]pyrimidin-4-one of Formula III or its salt with 6-fluoro-3-(4-piperidinyl)-1,2-benzisoxazole Formula II or its salt to afford (±)-3-[2-[4-(6-fluoro-1,2-benzisoxazol-3-yl)-1-piperidinyl]ethyl]-6,7,8,9-tetrahydro-9-hydroxy-2-methyl-4H-pyrido[1,2-a]pyrimidin-4-one of Formula I.

In another embodiment, the present invention provides processes for the purification of paliperidone, an embodiment comprising:

(1) providing a solution of paliperidone in a suitable solvent or mixture of solvents;

(2) cooling the solution of step (1); and

(3) recovering the solid formed in step (2) to afford the compound of Formula I.

In another embodiment, the present invention provides substantially pure paliperidone.

In yet another embodiment, the present invention provides paliperidone substantially free of risperidone and a 9-oxo impurity. In another embodiment, paliperidone substantially free of risperidone and a 9-oxo impurity may be obtained by the above processes of the present invention.

The processes of the present invention are simple, cost effective, and eco-friendly, affording high yields and purity, and are well-suited for use on a commercial scale.

### BRIEF DESCRIPTION OF THE DRAWINGS

FIG. 1 is a schematic representation of a process for the preparation of paliperidone of Formula I.

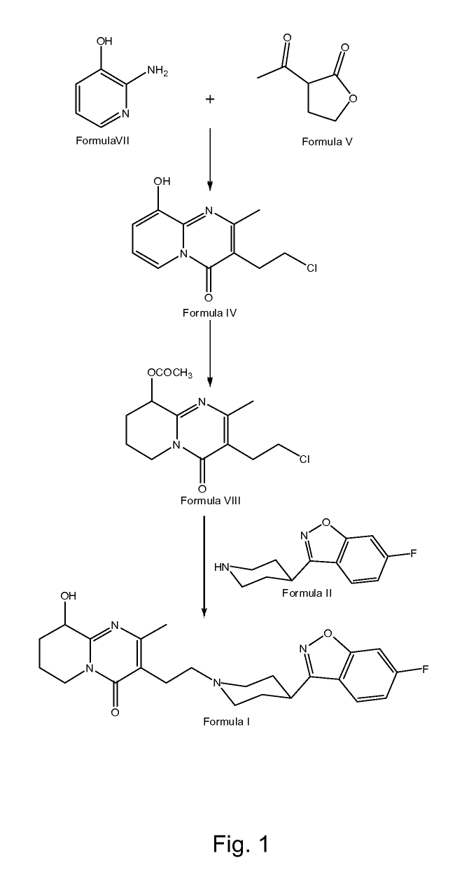

FIG. 2 is a schematic representation of an alternative process for the preparation of paliperidone of Formula I.

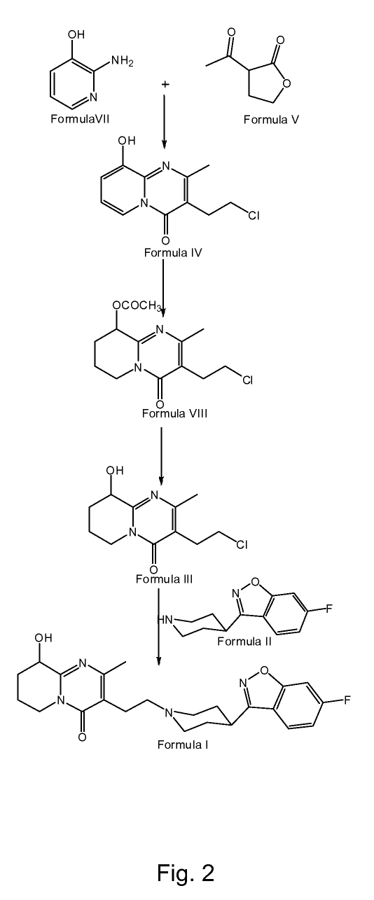

FIG. 3 is a schematic representation of an alternative process for the preparation of 3-(2-chloroethyl)-6,7,8,9-tetrahydro-9-acetyloxy-2-methyl-4H-pyrido[1,2-a]pyrimidin-4-one of Formula VIII.

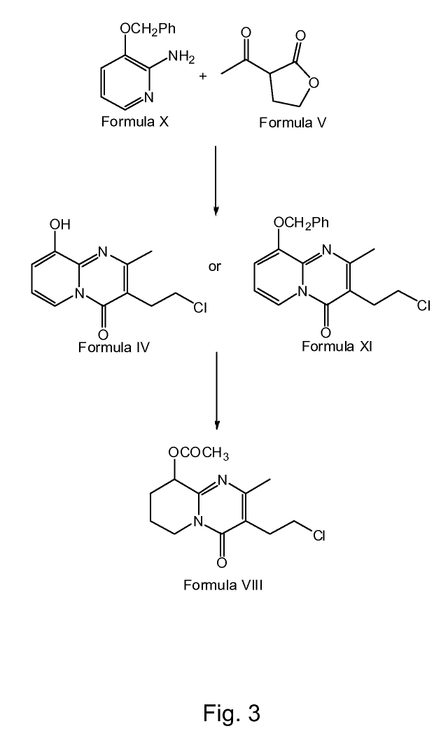

FIG. 4 is an X-ray powder diffraction (XRPD) pattern of a sample of paliperidone, as prepared in Example 10.

### DETAILED DESCRIPTION

The present application relates to improved processes for preparing paliperidone of Formula I. An embodiment of a process comprises:

(1) reacting 3-hydroxy-2-pyridinamine of Formula VII with 3-acetyl-4,5-dihydro-2(3H)-furanone of Formula V to afford 3-(2-chloroethyl)-2-methyl-9-hydroxy-4H-pyrido[1,2-a]pyrimidin-4-one of Formula IV;

(2) reducing 3-(2-chloroethyl)-2-methyl-9-hydroxy-4H-pyrido[1,2-a]pyrimidin-4-one of Formula IV in the presence acetic anhydride to afford 3-(2-chloroethyl)-6,7,8,9-tetrahydro-9-acetyloxy-2-methyl-4H-pyrido[1,2-a]pyrimidin-4-one of Formula VIII and optionally converting the compound VIII into its salt; and

(3) condensing 3-(2-chloroethyl)-6,7,8,9-tetrahydro-9-acetyloxy-2-methyl-4H-pyrido[1,2-a]pyrimidin-4-one of Formula VIII or its salt with 6-fluoro-3-(4-piperidinyl)-1,2-benzisoxazole of Formula II or its salt, to afford (±)-3-[2-[4-(6-fluoro-1,2-benzisoxazol-3-yl)-1-piperidinyl]ethyl]-6,7,8,9-tetrahydro-9-hydroxy-2-methyl-4H-pyrido[1,2-a]pyrimidin-4-one of Formula I.

Step (1) involves reaction of the compounds of Formula VII and Formula V using cyclizing agents in the presence of organic solvent(s) to afford the compound of Formula IV.

Useful cyclizing agents include but are not limited to phosphorous oxychloride, thionyl chloride, phosphorous trichloride, phosphoryl bromide and sulfuryl chloride.

The reaction may be carried out in the presence or absence of a solvent. Useful solvents include, but are not limited to: halogenated hydrocarbons such as dichloromethane, dichloroethane, chloroform, and the like; hydrocarbons such as toluene, xylene, n-Hexane, cyclohexane, n-heptane, and the like; ketones such as acetone, methyl isobutyl ketone, methyl ethyl ketone, and the like; esters such as ethyl acetate, n-propyl acetate, isopropyl acetate and the like; and mixtures thereof.

The reaction may be conducted at temperatures ranging from about 10° C. to the reflux temperature of the solvent used.

The reaction may be carried out for any desired time periods to achieve the desired product yield and purity. The reaction times vary from about 30 minutes to about 10 hours, or longer.

Isolation of the solid product thus obtained includes collection of the material by any techniques such as decantation, filtration by gravity or suction, centrifugation, and the like, and optional washing with the solvent. If required, the reaction mass may be cooled before product isolation.

The solid material obtained by any of the techniques described above may be further dried. Drying may be suitably carried out by any methods such as use of a tray dryer, vacuum oven, air oven, fluidized bed drier, spin flash dryer, flash dryer, and the like. The drying may be carried out under reduced pressures and at various temperatures. The temperatures may range from about ambient temperature to about 100° C., for a time period that produces the desired result.

Optionally, the compound of Formula IV may be converted into its acid-addition salt by reacting it with a pharmaceutically acceptable acid. Examples of such acids include: inorganic acids such as hydrochloric acid, sulfuric acid, phosphoric acid, hydrobromic acid, and the like; and organic acids such as oxalic acid, maleic acid, fumaric acid, malic acid, tartaric acid, citric acid, benzoic acid, and the like. The conversion of the compound of Formula IV into its salt increases the stability of the compound and hence these salts may be stored for an extended time depending on their stability after their manufacture.

Step 2) involves reduction of the compound of Formula IV using a suitable reducing agent in the presence acetic anhydride, in a suitable solvent, at suitable temperatures to afford the compound of Formula VIII.

The compound of Formula VIII in step 2 may be produced by catalytically reducing the compound of Formula IV in a hydrogen atmosphere in the presence of a catalyst. Catalysts that can be used for the reduction include, for example, platinum oxide, platinum on activated carbon, palladium on activated carbon, palladium on barium sulfate, palladium on calcium carbonate, palladium on barium carbonate, copper-chromium oxide, rhodium, cobalt, ruthenium, etc. The amount of the catalyst used may be about 1 to 100, or about 5-100, percent by weight of the amount of the compound having Formula IV.

The reaction may be advantageously conducted in a solvent inert to the reaction. The solvents include, but are not limited to: ketones such as acetone, methyl ethyl ketone, methyl isobutyl ketone, and the like; halogenated hydrocarbons such as dichloromethane, dichloroethane, chloroform, and the like; hydrocarbons such as toluene, xylene, cyclohexane and the like; esters such as ethyl acetate, isopropyl acetate, tertiary-butyl acetate and the like; acetic acid; and mixtures thereof in various proportions.

Although U.S. Pat. No. 5,158,952 describes a reduction of 3-(2-chloroethyl)-2-methyl-9-(phenylmethoxy)-4H-pyrido[1,2-a]pyrimidin-4-one involving the use of palladium on charcoal in methanol, repeating the experiment has not provided the results reported therein.

The reaction time varies, depending on the activity of the catalyst and the amount thereof used. In general, it is about 30 minutes to 24 hours, or about 30 minutes to 6 hours. The reaction temperatures are generally in the range of about 0 to 120° C., or about 20 to 80° C. The pressures for conducting the reaction are generally about 1 to 100 atmospheres. Additives (promoters) that enhance the activity of the catalyst used can be added to the reaction system. Acidic additives advantageously useful for this purpose include, for example: inorganic acids such as hydrochloric acid, sulfuric acid, nitric acid, perchloric acid, hydrobromic acid, phosphoric acid, etc.; and organic acids such as acetic acid, trifluoroacetic acid, oxalic acid, phthalic acid, fumaric acid, tartaric acid, citric acid, succinic acid, methanesulfonic acid, p-toluenesulfonic acid, 10-camphorsulfonic acid, etc. Basic additives are also advantageously useful and include, for example, sodium hydroxide, potassium hydroxide, ammonia, sodium acetate, triethyl phosphonium acetate, etc.

The product of Formula VIII may be used in the next reaction step, without isolation from the reaction mixture or in the form of a crude product. If desired, however, it may be isolated from the reaction mixture by ordinary methods, and it can be easily purified by any means of separation, for example, recrystallization, distillation and chromatography.

After reaction completion, the reaction mass is optionally filtered and optionally washed with the same solvent.

The compound may be isolated by removal of the solvent. The solvent may be removed using any suitable methods such as evaporation, atmospheric distillation, or distillation under vacuum.

Distillation of the solvent may be conducted under vacuum, such as below about 100 mm Hg to below about 600 mm Hg, at elevated temperatures such as about 20° C. to about 70° C. Any temperature and vacuum conditions may be used as long as they do not influence the nature of the product. The vacuum and the temperatures used for the removal of the solvent depend on parameters such as the boiling point of the solvent and may readily be determined by persons skilled in the art.

The compound may be isolated by addition of water to the filtrate and extraction into a water-immiscible solvent such as dichloromethane, dichloroethane, chloroform, toluene, xylene, ethyl acetate, etc. Optionally, isolation may involve both of the above steps.

The solvent may be removed from the reaction mass according to the methods described above.

Optionally the compound of Formula VIII may be converted into its acid-addition salt by reacting it with a pharmaceutically acceptable acid. Examples of such acids include: inorganic acids such as hydrochloric acid, sulfuric acid, phosphoric acid, hydrobromic acid, and the like; and organic acids such as oxalic acid, maleic acid, fumaric acid, malic acid, tartaric acid, citric acid, benzoic acid, and the like. The conversion of the compound of Formula VIII into its salt increases the stability of the compound and hence these salts may be stored for an extended time, depending on their stability after their manufacture.

Alternatively, 3-(2-chloroethyl)-6,7,8,9-tetrahydro-9-acetyloxy-2-methyl-4H-pyrido[1,2-a]pyrimidin-4-one of Formula VIII may be prepared by a process which comprises:

(a) reacting 3-(phenylmethoxy)-2-pyridinamine of Formula X with 3-acetyl-4,5-dihydro-2(3H)-furanone of Formula V using suitable reagents in the presence of suitable organic solvent(s) at suitable temperatures to afford 3-(2-chloroethyl)-2-methyl-9-hydroxy-4H-pyrido[1,2-a]pyrimidin-4-one of Formula IV or 3-(2-chloromethyl)-2-methyl-9-(phenylmethoxy)-4H-pyrido[1,2a]pyrimidine-4-one of Formula XI;

(b) reducing 3-(2-chloroethyl)-2-methyl-9-hydroxy-4H-pyrido[1,2-a]pyrimidin-4-one of Formula IV or 3-(2-chloromethyl)-2-methyl-9-(phenylmethoxy)-4H-pyrido[1,2a]pyrimidine-4-one of Formula XI in the presence acetic anhydride to afford 3-(2-chloroethyl)-6,7,8,9-tetrahydro-9-acetyloxy-2-methyl-4H-pyrido[1,2-a]pyrimidin-4-one of Formula VIII and optionally converting the compound VIII into its salt.

3-(2-chloroethyl )-2-methyl-9-hydroxy-4H-pyrido[1,2-a]pyrimidin-4-one of Formula IV or 3-(2-Chloromethyl)-2-methyl-9-(phenylmethoxy)-4H-pyrido[1,2a]pyrimidine-4-one of Formula XI may be prepared by reacting 3-(phenylmethoxy)-2-pyridinamine with 3-acetyl-4,5-dihydro-2(3H)-furanone. The conditions, solvents, and the isolation techniques may be carried out as described above for the preparation of Formula IV.

3-(2-chloroethyl)-6,7,8,9-tetrahydro-9-acetyloxy-2-methyl-4H-pyrido[1,2-a]pyrimidin-4-one of Formula VIII may be prepared by the reduction of 3-(2-chloroethyl)-2-methyl-9-hydroxy-4H-pyrido[1,2-a]pyrimidin-4-one of Formula IV or 3-(2-Chloromethyl)-2-methyl-9-(phenylmethoxy)-4H-pyrido[1,2a]pyrimidine-4-one of Formula XI in the presence acetic anhydride. The resulting compound of Formula VIII may be optionally converted into its salt. The conditions, solvents, quantities of raw materials and the isolation techniques may be carried out as described above for the preparation of Formula VIII.

Step 3) involves condensation of the compound of Formula VIII with the compound of Formula II, to afford the compound of Formula I.

In an aspect, the present application provides processes for preparing paliperidone, an embodiment of which comprises condensation of 3-(2-chloroethyl)-6,7,8,9-tetrahydro-9-acetyloxy-2-methyl-4H-pyrido[1,2-a]pyrimidin-4-one of Formula VIII or its salt with 6-fluoro-3-(4-piperidinyl)-1,2-benzisoxazole Formula II or its salt, by subjecting the reaction mixture to microwave radiation.

The reaction may be conducted in the presence or absence of a base. Bases that are useful in the reaction include, but are not limited to: inorganic bases such as alkali metal or alkaline earth metal carbonates, hydrogen carbonates, hydroxides, oxides, carboxylates, and alkoxides, e.g., sodium carbonate, sodium hydrogen carbonate, potassium carbonate, sodium hydroxide, calcium oxide, sodium acetate, sodium methoxide, and the like; and organic bases such as, for example, tertiary amines, e.g., N,N-diethylethanamine, N-(1-methylethyl)-2-propanamine, 4-ethylmorpholine, 1,4-diazabicyclo[2.2.2]-octane, N-methyl morpholine, diisopropyl ethyl amine, pyridine, and the like. Addition of an iodide salt, such as an alkali metal iodide, may facilitate the reaction. Further, the reaction may be conducted in the presence of a catalyst such as a crown ether, 1,4,7,10,13,16-hexaoxacyclooctadecane. Stirring and elevated temperatures may enhance the rate of the reaction; in particular, the reaction may be conducted at the reflux temperature of the reaction mixture. Additionally, it may be advantageous to conduct this step under an inert atmosphere such as, for example, oxygen-free argon or nitrogen gas. The base is used in an amount of approximately 1 to 10 moles, or approximately 1 to 5 moles, per mole of the compound of Formula (VIII).

The solvents that may be utilized for this step include, but are not limited to: alcohols such as methanol, ethanol, isopropyl alcohol and n-propanol; halogenated hydrocarbons such as dichloromethane, 1,2-dichloroethane, chloroform, and carbon tetrachloride; ketones such as acetone, ethyl methyl ketone and methyl isobutyl ketone; esters such as ethyl acetate, n-propyl acetate, n-butyl acetate, isobutyl acetate and t-butyl acetate; ethers such as diethyl ether, dimethylether, di-isopropylether, methyl tertiary-butyl ether, 1,1′-oxybisethane, tetrahydrofuran and 1,4-dioxane; hydrocarbons such as n-heptane, cyclohexane and n-hexane; aromatic solvents, e.g. benzene, toluene, xylene, chlorobenzene, and methoxybenzene; nitriles such as acetonitrile and propionitrile; dimethylsulfoxide (DMSO), N,N-dimethylformamide (DMF) and N,N-dimethylacetamide; pyridine; 1,3-dimethyl-3,4,5,6-tetrahydro-2(1H)-pyrimidinone; 1,3-dimethyl-2-imidazolidinone; 1,1,3,3-tetramethyl urea; 1-methyl-2-pyrrolidinone; nitrobenzene; water; and mixtures thereof.

The reaction may be carried out for any desired time periods to achieve the desired product yield and purity. Typical reaction times can vary from about 20 hours to 50 hours, or longer.

After the reaction is completed, the solvent may be removed using any suitable method such as evaporation, atmospheric distillation, or distillation under vacuum.

Distillation of the solvent may be conducted under vacuum, such as below about 100 mm Hg or below about 600 mm Hg at elevated temperatures such as about 20° C. to about 70° C. Any temperature and vacuum conditions may be used as long as they do not influence the nature of the product. The vacuum and the temperature used for the removal of the solvent depend on parameters such as the boiling point of the solvent and may be readily determined by persons skilled in the art.

The reaction mass may be diluted by addition of water and extracted into a water immiscible solvent such as dichloromethane, dichloroethane, chloroform, toluene, xylene, ethyl acetate etc. Optionally, isolation may involve both of the above steps.

The solvent may be removed from the reaction mass using methods described above.

The compound may be isolated by adding suitable solvents such as acetone, methyl ethyl ketone, isopropyl alcohol, water, n-hexane, n-heptane, etc.

The reaction mass may be optionally heated to about 40 to 50° C., or the reflux temperature of the solvent. Isolation of the product thus obtained includes collection of the material by any techniques such as filtration by gravity or suction, centrifugation, and the like and optional washing with the solvent. If desired, the reaction mass may be cooled before isolation.

The solid material obtained by any of the techniques described above may be further dried. Drying may be suitably carried out by any methods such as using a tray dryer, vacuum oven, air oven, fluidized bed drier, spin flash dryer, flash dryer, and the like. The drying may be carried out under reduced pressures and at elevated temperatures. The temperatures may range from about ambient temperature to about 100° C., for a time period that produces the desired result.

The compound of Formula I has basic properties and, consequently, it may be converted to therapeutically active non-toxic acid addition salt forms by treatment with appropriate acids, such as, for example: inorganic acids, such as a hydrohalic acid, e.g. hydrochloric or hydrobromic acid and the like, sulfuric acid, nitric acid, phosphoric acid and the like; or an organic acid such as, for example, acetic, propanoic, hydroxyacetic, 2-hydroxypropanoic, 2-oxopropanoic, ethanedioic, propanedioic, butanedioic, (Z)-2-butenedioic, (E)-2-butenedioic, 2-hydroxybutanedioic, 2,3-dihydroxybutanedioic, 2-hydroxy-1,2,3-propanetricarboxylic, methanesulfonic, ethanesulfonic, benzenesulfonic, 4-methylbenzenesulfonic, 2-hydroxybenzoic, 4-amino-2-hydroxybenzoic acids, and the like. Conversely, a salt form may be converted into the free base form by treatment with an alkali.

The present application also relates to alternative processes for the preparation of paliperidone of Formula I. An embodiment of an alternate process comprises:

(1) converting 3-(2-chloroethyl)-6,7,8,9-tetrahydro-9-acetyloxy-2-methyl-4H-pyrido[1,2-a]pyrimidin-4-one of Formula VIII to 3-(2-chloroethyl)-6,7,8,9-tetrahydro-9-hydroxy-2-methyl-4H-pyrido[1,2-a]pyrimidin-4-one of Formula III by hydrolysis and optionally converting the compound of Formula III into its salt; and

(2) condensing 3-(2-chloroethyl)-6,7,8,9-tetrahydro-9-hydroxy-2-methyl-4H-pyrido[1,2-a]pyrimidin-4-one of Formula III or its salt with 6-fluoro-3-(4-piperidinyl)-1,2-benzisoxazole Formula II or its salt, to afford (±)-3-[2-[4-(6-fluoro-1,2-benzisoxazol-3-yl)-1-piperidinyl]ethyl]-6,7,8,9-tetrahydro-9-hydroxy-2-methyl-4H-pyrido[1,2-a]pyrimidin-4-one of Formula I.

Step 1) involves the hydrolysis of 3-(2-chloroethyl)-6,7,8,9-tetrahydro-9-acetyloxy-2-methyl-4H-pyrido[1,2-a]pyrimidin-4-one of Formula VIII to afford 3-(2-chloroethyl)-6,7,8,9-tetrahydro-9-hydroxy-2-methyl-4H-pyrido[1,2-a]pyrimidine-4-one of Formula III, and optionally converting it into a salt

3-(2-chloroethyl)-6,7,8,9-tetrahydro-9-acetyloxy-2-methyl-4H-pyrido[1,2-a]pyrimidin-4-one of Formula VIII used in this step may be prepared according to the methods described in this application or other methods known in the art.

The hydrolysis step may be carried out in an acidic or basic medium. An inorganic basic compound such as calcium carbonate, sodium carbonate, sodium hydroxide, sodium hydrogen carbonate and the like may be used for this step. Examples of useful inorganic acids include hydrochloric acid, sulfuric acid, phosphoric acid, hydrobromic acid and the like. Any other methods that accomplish the hydrolysis without affecting the product molecule may also be used.

The base or acid that is used in this reaction may be used in amounts ranging from approximately 1 to 10 moles, or approximately 1 to 5 moles, per mole of compound of Formula VIII.

The solvents that may be utilized for this hydrolysis include, but are not limited to: alcohols such as methanol, ethanol, isopropyl alcohol and n-propanol; ketones such as acetone, ethyl methyl ketone and methyl isobutyl ketone; ethers such as diethyl ether, dimethylether, di-isopropylether, methyl tertiary-butyl ether, 1,1′-oxybisethane, tetrahydrofuran and 1,4-dioxane; hydrocarbons such as n-heptane, cyclohexane and n-hexane; aromatic solvents, e.g., toluene, xylene, chlorobenzene, methoxybenzene; water; and mixtures thereof.

The reaction may be carried out from about room temperature to about the reflux temperature of the solvent used.

The reaction may be carried out for any desired time periods to achieve the desired product yield and purity. The reaction times typically vary from about 30 minutes to 10 hours, or longer.

Isolation of the product thus obtained includes collection of the material using any techniques such as decantation, filtration by gravity or suction, centrifugation, and the like and optional washing with the solvent. If desired, the reaction mass may be cooled before product isolation.

The solid material obtained by any of the techniques described above may be further dried. Drying may be suitably carried out by any methods involving equipment such as a tray dryer, vacuum oven, air oven, fluidized bed drier, spin flash dryer, flash dryer and the like. The drying may be carried out under reduced pressures and at temperatures. The temperature may range from about ambient temperature to about 100° C., for any time period that produces the desired result.

Optionally the compound of Formula III may be converted into its acid-addition salt by reacting it with a pharmaceutically acceptable acid. Examples of useful acids include: inorganic acids such as hydrochloric acid, sulfuric acid, phosphoric acid, hydrobromic acid and the like; and organic acids such as oxalic acid, maleic acid, fumaric acid, malic acid, tartaric acid, citric acid, benzoic acid and the like. The conversion of the compound of Formula III into its salt increases the stability of the compound and hence these salts may be stored for extended times, depending on their stability after their manufacture.

Step 2) involves condensation of the 3-(2-chloroethyl)-6,7,8,9-tetrahydro-9-hydroxy-2-methyl-4H-pyrido[1,2-a]pyrimidin-4-one of Formula III or its salt with 6-fluoro-3-(4-piperidinyl)-1,2-benzisoxazole Formula II or its salt, to afford the compound of Formula I.

In another aspect, the present application provides processes for preparing paliperidone, an embodiment of which comprises condensation of 3-(2-chloroethyl)-6,7,8,9-tetrahydro-9-hydroxy-2-methyl-4H-pyrido[1,2-a]pyrimidin-4-one of Formula III or its salt with 6-fluoro-3-(4-piperidinyl)-1,2-benzisoxazole Formula II or its salt, including subjecting the reaction mixture to microwave radiation.

The reaction may be conducted in the presence or absence of a base. Bases that are useful in the reaction include but are not limited to:, inorganic bases such as alkali metal or alkaline earth metal carbonates, hydrogen carbonates, hydroxides, oxides, carboxylates, and alkoxides, e.g., sodium carbonate, sodium hydrogen carbonate, potassium carbonate, sodium hydroxide, calcium oxide, sodium acetate, sodium methoxide, and the like; or organic bases such as, for example, tertiary-amines, e.g. N,N-diethylethanamine, N-(1-methylethyl)-2-propanamine, 4-ethylmorpholine, 1,4-diazabicyclo[2.2.2]-octane, N-methyl morpholine, diisopropyl ethyl amine, pyridine and the like. Addition of an iodide salt, such as an alkali metal iodide, may facilitate the reaction. Further, the reaction may be conducted in the presence of a catalyst such as a crown ether, 1,4,7,10,13,16-hexaoxacyclooctadecane. Stirring and elevated temperatures may enhance the rate of the reaction; in particular the reaction may be conducted at the reflux temperature of the reaction mixture. Additionally, it may be advantageous to conduct this step under an inert atmosphere such as, for example, oxygen-free argon or nitrogen gas. The base is used in an amount of approximately 1 to 10 moles, or approximately 1 to 5 moles, per mole of compound of Formula III.

The solvents that may be utilized for this step include, but are not limited to, alcohols such as methanol, ethanol, isopropyl alcohol and n-propanol; halogenated hydrocarbons such as dichloromethane, 1,2-dichloroethane, chloroform and carbon tetrachloride; ketones such as acetone, ethylmethyl ketone and methyl isobutyl ketone; esters such as ethyl acetate, n-propyl acetate, n-butyl acetate, isobutyl acetate and t-butyl acetate; ethers such as diethyl ether, dimethyl ether, di-isopropyl ether, methyl tertiary-butyl ether, 1,1′-oxybisethane, tetrahydrofuran and 1,4-dioxane; hydrocarbons such as n-heptane, cyclohexane and n-hexane; aromatic solvents, e.g., benzene, toluene, xylene, chlorobenzene, and methoxybenzene; nitriles such as acetonitrile and propionitrile; dimethylsulfoxide (DMSO); N,N-dimethylformamide (DMF); N,N-dimethylacetamide; pyridine; 1,3-dimethyl-3,4,5,6-tetrahydro-2(1H)-pyrimidinone; 1,3-dimethyl-2-imidazolidinone; 1,1,3,3-tetramethyl urea; 1-methyl-2-pyrrolidinone; nitrobenzene; water; and mixtures thereof.

The reaction may be carried out for any desired time periods to achieve the desired product yield and purity. Typically, reaction times vary from about 20 hours to 50 hours, or longer.

The reaction when carried out in the presence of microwave radiation may be maintained until the completion of the reaction. The reaction times may range from about 1 minute to 5 hours, or longer. The temperatures during the reaction may range from room temperature to the reflux temperature of the solvent.

After the completion of the reaction, the solvent may be removed using any suitable method such as evaporation, atmospheric distillation, or distillation under vacuum.

Distillation of the solvent may be conducted under vacuum, such as below about 100 mm Hg or below about 600 mm Hg at elevated temperatures such as about 20° C. to about 70° C. Any temperature and vacuum conditions may be used as long as they do not influence the nature of the product. The vacuum and the temperature used for the removal of the solvent depend on parameters such as the boiling point of the solvent and may be readily determined by persons skilled in the art.

The compound may be diluted by addition of water and extracted into a water-immiscible solvent such as dichloromethane, dichloroethane, chloroform, toluene, xylene, ethyl acetate, etc. Optionally, isolation may involve both the steps.

The solvent may be removed from the reaction mass using any of the methods described above.

The compound may be isolated by adding suitable solvents such as acetone, methyl ethyl ketone, isopropyl alcohol, water, n-hexane, n-heptane, etc.

The reaction mass may be optionally heated to about 40 to 50° C., or the reflux temperature of the solvent. Isolation of the product thus obtained is accomplished using methods including collection of the material by any techniques such as filtration by gravity or suction, centrifugation, and the like and optional washing with the solvent. If required, the reaction mass may be cooled before isolation.

The solid material obtained by any of the techniques described above may be further dried. Drying may be suitably carried out in any equipment such as a tray dryer, vacuum oven, air oven, fluidized bed drier, spin flash dryer, flash dryer and the like. The drying may be carried out under reduced pressures and at elevated temperatures. The temperatures may range from about ambient temperature to about 100° C., for a time period that produces the desired result.

The compound of Formula I has basic properties and consequently, it may be converted to therapeutically active non-toxic acid addition salt forms by treatment with appropriate acids, for example: inorganic acids, such as hydrohalic acids, e.g. hydrochloric and hydrobromic acid, and the like, sulfuric acid, nitric acid, phosphoric acid and the like; or organic acids, such as, for example, acetic, propanoic, hydroxyacetic, 2-hydroxypropanoic, 2-oxopropanoic, ethanedioic, propanedioic, butanedioic, (Z)-2-butenedioic, (E)-2-butenedioic, 2-hydroxybutanedioic, 2,3-dihydroxybutanedioic, 2-hydroxy-1,2,3-propanetricarboxylic, methanesulfonic, ethanesulfonic, benzenesulfonic, 4-methylbenzenesulfonic, 2-hydroxybenzoic, 4-amino-2-hydroxybenzoic acids, and the like. Conversely, the salt forms may be converted into the free base form by treatment with alkali.

In another aspect, the present invention provides processes for the purification of paliperidone, an embodiment comprising:

(1) providing a solution of paliperidone;

(2) cooling the solution of step (1); and

(3) recovering a solid formed in step (2) to afford the compound of Formula I.

Step (1) involves providing a solution of paliperidone in a solvent.

The solvents that may be utilized for this step include, but are not limited to, alcohols such as methanol, ethanol, isopropyl alcohol and n-propanol; halogenated hydrocarbons such as dichloromethane, 1,2-dichloroethane, chloroform and carbon tetrachloride; ketones such as acetone, ethyl methyl ketone and methyl isobutyl ketone; esters such as ethyl acetate, n-propyl acetate, n-butyl acetate, isobutyl acetate and t-butyl acetate; ethers such as diethyl ether, dimethyl ether, di-isopropyl ether, methyl tertiary-butyl ether, 1,1′-oxybisethane, tetrahydrofuran and 1,4-dioxane; aromatic solvents, e.g., toluene, xylene, chlorobenzene, and methoxybenzene; nitriles such as acetonitrile and propionitrile; dimethylsulfoxide (DMSO); N,N-dimethylformamide (DMF); N,N-dimethylacetamide; pyridine; 1,3-dimethyl-3,4,5,6-tetrahydro-2(1H)-pyrimidinone; 1,3-dimethyl-2-imidazolidinone; 1,1,3,3-tetramethylurea; 1-methyl-2-pyrrolidinone; nitrobenzene; and mixtures thereof.

Suitable temperatures for dissolution of the compound in a solvent may range from about 10° C. to about the reflux temperature of the solvent.

Stirring may be continued for any desired time periods to achieve a complete dissolution of the compound. The stirring time may range from about 5 minutes to 3 hours, or longer.

The solution may be optionally treated with carbon and filtered to get a particle-free solution.

Optionally the solution may be obtained directly from a reaction mass.

Step (2) involves optional cooling of the solution obtained in step (1) to precipitate the compound.

The temperatures for this cooling step may range from about −5° C. to about 50° C. Stirring may be continued from about 30 minutes to 3 hours, or longer, to achieve a more complete crystallization of the compound. In an alternate embodiment, the isolation of the product from the solution of step (1) may also be promoted by the addition of an anti-solvent.

The anti-solvents that may be used for this step include, but are not limited to, hydrocarbon solvents such as n-heptane, cyclohexane and n-hexane, water, and mixtures thereof.

Step (3) involves recovering the solid of step (2) by any methods to afford the compound.

The method, by which the solid material is recovered from the final mixture, may involve any of techniques such as filtration by gravity or by suction, centrifugation, and the like. The crystals so isolated will carry a small proportion of occluded mother liquor containing a higher percentage of impurities. If desired, the crystals may be washed with a solvent to wash out the mother liquor.

The wet cake obtained in step (3) may be further dried. Drying may be suitably carried out in equipment such as a tray dryer, vacuum oven, air oven, fluidized bed drier, spin flash dryer, flash dryer and the like. The drying may be carried out at temperatures from about 35° C. to about 100° C., with or without vacuum. The drying may be carried out for any desired time until the required product quality is achieved. The drying time may vary from about 1 to about 20 hours, or longer.

In the event that a higher purity is required, the above recrystallization process may be repeated one, two, or more times.

The paliperidone of Formula I obtained according to the processes of the present invention may be further purified by slurrying in a solvent. The solvents that may be utilized for this purification step are the same as the solvents that are discussed in the earlier purification step.

In another embodiment, the present application is relates to substantially pure paliperidone.

In yet another embodiment, the present application provides paliperidone substantially free of risperidone and a 9-oxo impurity.

As used herein “substantially pure” refers to chemical purity. Paliperidone of the present application contains less than about 0.5% of total impurities, or less than about 0.1% of total impurities, by weight.

The possible impurities in paliperidone include risperidone, 9-oxo impurity, raw materials, intermediates and other process-related impurities. In embodiments, paliperidone obtained by the present application contains less than about 0.1% by weight of the individual impurities. These impurities can be characterized by high performance liquid chromatography (“HPLC”).

A chemical name for risperidone is 3-[2-[4-(6-fluoro-1,2-benzisoxazol-3-yl)-1-piperidinyl]ethyl]-6,7,8,9-tetrahydro-2-methyl-4H-pyrido[1,2-a]pyrimidin-4-one and is it structurally represented by Formula VI.

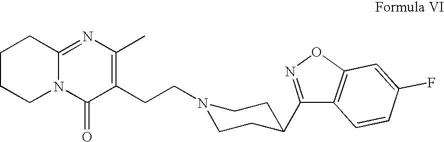

A chemical name for a 9-oxo impurity is (±)-3-[2-[4-(6-fluoro-1,2-benzisoxazol-3-yl)-1-piperidinyl]ethyl]-6,7,8,9-tetrahydro-9-oxo-2-methyl-4H-pyrido[1,2-a]pyrimidin-4-one and it is structurally represented by Formula IX.

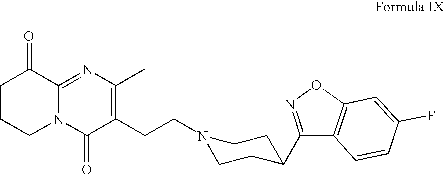

Other possible process related impurities during the synthesis of the paliperidone apart from the raw materials are described below as Impurity A, Impurity B, Impurity C, and Impurity D, having the following structures:

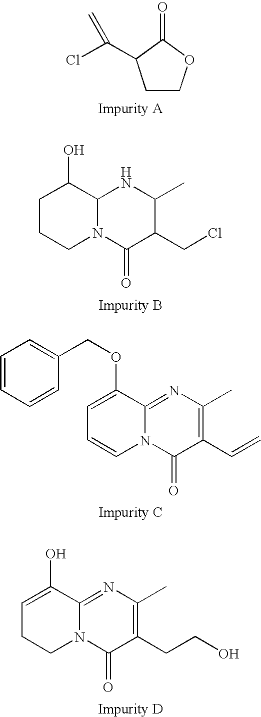

An example of a HPLC method that can be used for the analysis of paliperidone includes an Inertsil ODS 3V, 250×4.6 mm, 5 μm or equivalent column. Additional method parameters are given in the table below:

TABLE 1

| Flow rate | 1.0 ml/minute | | |
|---|---|---|---|
| Elution | Gradient | | |
| Wavelength | 210 nm | | |
| Injection volume | 10 μl | | |
| Column oven temperature | Ambient | | |
| Run time | 60 minutes | | |
| Diluent | Mobile phase B | | |
| Sample concentration | 0.5 mg/ml | | |
| Buffer preparation | Dissolve 2.72 g of KH₂PO₄ in 1000 ml of | | |
| | milli-q water, add 1.0 mL of triethyl | | |
| | amine and adjust pH to 6.0 with | | |
| | orthophosphoric acid. | | |
| Mobile phase | Mobile phase A: mix buffer and | | |
| | acetonitrile in the volume ratio 80:20. | | |
| | Mobile phase B: mix buffer and | | |
| | acetonitrile in the volume ratio 20:80. | | |
| | | Mobile | Mobile Phase |
| Gradient program | Time | Phase A | B |
| | 00.00 | 90 | 10 |
| | 20.00 | 90 | 10 |
| | 30.00 | 60 | 40 |
| | 40.00 | 40 | 60 |
| | 50.00 | 60 | 40 |
| | 55.00 | 70 | 30 |
| | 60.00 | 90 | 10 |

The crystalline paliperidone in the currently marketed INVEGA™ products is characterized by XRPD peaks at about 7.5, 8.2, 10.3, 12.5, 13.2, 13.8, 14.6, 15.0, 16.2, 18.7, 19.3, 20.1, 20.7, 22.1, 24.7, and 25.1, ±0.2 degrees as obtained using copper Kα-radiation. In an aspect, paliperidone obtained by the processes of the present invention provides a crystalline polymorphic form which has similar characteristic XRPD peaks.

The paliperidone of Formula I obtained according to the processes of the present application may be milled to get a desired particle size distribution.

In the foregoing description, certain conditions such as temperatures and certain operations such as filtration have been described to illustrate the invention. Persons skilled in the art will be aware that other conditions and operations will also be suitable, and those are included within the invention. For example, operations such as centrifugation, decantation, etc. are acceptable substitutes for filtration.

Another aspect of the present invention provides pharmaceutical compositions containing a therapeutically effective amount of pure paliperidone or a pharmaceutically acceptable salt thereof, containing less than about 0.25% by weight of any individual impurity, together with one or more pharmaceutically acceptable excipients.

The pharmaceutical compositions comprising paliperidone or its pharmaceutically acceptable salt of the invention along with one or more pharmaceutically acceptable excipients may be formulated as: solid oral dosage forms such as, but not limited to, powders, granules, pellets, tablets, and capsules; liquid oral dosage forms such as but not limited to syrups, suspensions, dispersions, and emulsions; and injectable preparations such as but not limited to solutions, dispersions, and freeze dried compositions. Formulations may be in the form of immediate release, delayed release or modified release. Further, immediate release compositions may be conventional, dispersible, chewable, mouth dissolving, or flash melt preparations, and modified release compositions may comprise hydrophilic or hydrophobic, or combinations of hydrophilic and hydrophobic, release rate-controlling substances to form matrix or reservoir systems or combinations of matrix and reservoir systems. The compositions may be prepared using any one or more of direct blending, dry granulation, wet granulation, and extrusion and spheronization. Compositions may be presented as uncoated, film coated, sugar coated, powder coated, enteric coated or modified release coated.

Compositions of the present invention may further comprise one or more pharmaceutically acceptable excipients.

Pharmaceutically acceptable excipients that are useful in the present invention include, but are not limited to, any one or more of: diluents such as starch, pregelatinized starch, lactose, powdered cellulose, microcrystalline cellulose, dicalcium phosphate, tricalcium phosphate, mannitol, sorbitol, sugar and the like; binders such as acacia, guar gum, tragacanth, gelatin, polyvinylpyrrolidones, hydroxypropyl celluloses, hydroxypropyl methylcelluloses, pregelatinized starches and the like; disintegrants such as starch, sodium starch glycolate, pregelatinized starches, crospovidones, croscarmellose sodium, colloidal silicon dioxide and the like; lubricants such as stearic acid, magnesium stearate, zinc stearate and the like; glidants such as colloidal silicon dioxide and the like; solubility or wetting enhancers such as anionic or cationic or neutral surfactants; complex forming agents such as various grades of cyclodextrins and resins; and release rate controlling agents such as hydroxypropyl celluloses, hydroxymethyl celluloses, hydroxypropyl methylcelluloses, ethylcelluloses, methylcelluloses, various grades of methyl methacrylates, waxes and the like. Other pharmaceutically acceptable excipients that are useful include but are not limited to film-formers, plasticizers, colorants, flavoring agents, sweeteners, viscosity enhancers, preservatives, antioxidants and the like.

Certain specific aspects and embodiments of the present invention will be explained in greater detail with reference to the following examples, which are provided by way of illustration only and should not be construed as limiting the scope of the invention in any manner.

### EXAMPLE 1

### Preparation of 3-(2-chloroethyl)-2-methyl-9-hydroxy-4H-pyrido[1,2-a]pyrimidin-4-one (Formula IV)

2-amino-3-hydroxypyridine (50 g), toluene (500 mL) and 2-acetyl butyrolactone (290 mL) were charged into a clean dry round bottom flask, cooled to 10° C. and POCl₃ (233 mL) was added to the reaction mass over 2 hours, 20 minutes at 10° C. The reaction mass was heated to 27° C. and then heated to 70° C. The reaction mass was maintained at 70° C. for 8 hours. After completion of the reaction, the reaction mass was poured into crushed ice (1500 g) followed by addition of water (500 mL). The reaction mass was stirred for 2 hours and layers were separated. The aqueous layer was washed with 2×250 mL of toluene. Separated aqueous layer was cooled to 5° C. and pH was adjusted to 8 with aqueous ammonia (800 mL) and the aqueous layer stirred for 2 hours at 10-20° C. The solid was filtered, washed with water (50 mL) and dried at 60° C. under vacuum for 15 hours to afford 82 g of the title compound.

### EXAMPLE 2

### Preparation of 3-(2-chloroethyl)-2-methyl-9-hydroxy-4H-pyrido[1,2-a]pyrimidin-4-one (Formula IV)

Phosphoryl chloride (210.8 g) was charged into a flask and cooled to about 5° C.-10° C. A solution of 2-amino-3-benzyloxypyridine (50 g) and 2-acetyl butyrolactone (160 g) in toluene (250 mL) was added to the above solution and then stirred for about 6 to about 8 hours at about 90° C. to about 95° C. After completion of the reaction, reaction mass was poured into crushed ice followed by addition of water. The reaction mass was then stirred for about 10 to about 15 minutes and the layers were separated. The aqueous layer was washed with toluene (2×150 mL) and the separated aqueous layer was cooled to about 10° C. to about 15° C. and the pH was adjusted to about 7 to about 8 with aqueous ammonia (400 mL) and stirred for about 30 to about 45 minutes. The solid was filtered, washed with water (200 mL). Charged wet solid and water (500 ml) into RBF and stirred at 25-35° C. for 30-40 minutes. The solid was filtered and washed with water (250 ml) and dried at about 50° C. under vacuum. The above obtained solid (38 g) and isopropyl alcohol (190 mL) were charged into a flask and stirred for about 45 minutes at reflux. The mixture was then cooled to about 25° C. to about 35° C. and stirred for about 60 minutes. The solid was filtered, washed with isopropyl alcohol (76 mL) and dried at about 50° C. under vacuum to afford the title compound (30 g).

### EXAMPLE 3

### Preparation of 3-(2-chloroethyl)-6,7,8,9-tetrahydro-9-acetyloxy-2-methyl-4H-pyrido[1,2-A]pyrimidin-4-one (Formula VIII)

3-(2-chloroethyl)-2-methyl-9-hydroxy-4H-pyrido[1,2-a]pyrimidin-4-one (70 g), acetic acid (1400 mL), acetic anhydride (70 mL) and 5% w/w palladium on carbon (35 g) were placed into a clean and dry autoclave vessel. To this reaction mass 6 kg/cm² hydrogen pressure was applied and the reaction mass was heated to 40° C. The reaction mass was maintained under agitation at 40° C. for 7 hours. After completion of the reaction, the reaction suspension was filtered and the solid washed with acetic acid (70 mL). The solvent was distilled completely from the filtrate at 50° C. under vacuum. To the residue water (700 mL) was added and the mixture was extracted with dichloromethane (3×350 mL). Combined organic layer was washed with water (2×350 mL) and saturated sodium chloride solution (350 mL). Organic layer was dried over anhydrous sodium sulphate and the solvent was distilled completely at 30° C. under vacuum to afford 75 g of the title compound.

### EXAMPLE 4

### Preparation of 3-(2-chloroethyl)-6,7,8,9-tetrahydro-9-hydroxy-2-methyl-4H-pyrido[1,2-a]pyrimidine-4-one (Formula III)

3-(2-chloroethyl)-6,7,8,9-tetrahydro-9-acetyloxy-2-methyl-4H-pyrido[1,2-a]pyrimidin-4-one (37.5 g), sodium hydroxide (6.3 g) and water (187.5 mL) were charged into a clean and dry flask and stirred. The reaction mass was stirred at 28° C. for 2 hours, 30 minutes. The solid was filtered, washed with water (37.5 mL) and dried at about 50° C. under vacuum for about 3 hours to afford 15 g of the title compound.

### EXAMPLE 5

### Preparation of 3-[2-[4-(6-fluoro-1,2-benzisoxazol-3-yl)-1-piperidinyl]ethyl]-6,7,8,9-tetrahydro-9-hydroxy-2-methyl-4H-pyrido[1,2-a]pyrimidin-4-one (Formula I)

3-(2-chloroethyl)-6,7,8,9-tetrahydro-9-acetyloxy-2-methyl-4H-pyrido[1,2-a]pyrimidin-4-one (37.5 g), 6-fluoro-3-(4-piperidinyl)-1,2-benzisoxazole monohydrochloride (25.3 g), methanol (375 mL) and N-(1-methylethyl)-2-propanamine (40 g) were charged into a clean and dry round bottom flask and stirred for about 40 hours at 60° C. After completion of the reaction, solvent was distilled completely under vacuum. To the residue water was added and extracted with dichloromethane (3×100 mL). The combined organic layer was washed with water and saturated sodium chloride solution. The solvent was distilled completely at about 40° C. under vacuum to afford a residue. To the above residue acetone (5 volumes to the weight of residue) was added and stirred for 1 hour, 15 minutes at about 25-35° C. The solid obtained was filtered, washed with acetone and dried at about 50° C. to a constant weight to afford 22.1 g of the title compound.

### EXAMPLE 6

### Preparation of 3-[2-[4-(6-fluoro-1,2-benzisoxazole-3-yl)-1-piperidinyl]ethyl]-6,7,8,9-tetrahydro-9-hydroxy-2-methyl-4H-pyrido[1,2-a]pyrimidin-4-one (Formula I)

3-(2-chloroethyl)-2-methyl-9-hydroxy-4H-pyrido[1,2-a]pyrimidin-4-one (18.5 g), 6-fluoro-3-(4-piperidinyl)-1,2-benzisoxazole monohydrochloride (19.5 g), methanol (185 mL) and N-(1-methylethyl)-2-propanamine (15.4 g) were charged into a clean and dry round bottom flask and stirred for about 36 hours at 60° C. After completion of the reaction, solvent was distilled completely under vacuum below 50° C. To the residue, water (100 mL) was added and the mixture was extracted with dichloromethane (3×100 mL). Combined organic layer was divided into two parts.

Part (i): The solvent was distilled completely at about 40° C. under vacuum to afford a residue. To the above residue was added acetone and the solvent was distilled under vacuum to remove the traces of dichloromethane. Acetone was added to the residue and cooled to 30° C., stirred for 50 minutes, filtered and washed with acetone. The compound was dried at about 58° C. until a constant weight was obtained. 10.6 g of the title compound was obtained.

Part (ii): The organic layer was refluxed for 10 minutes and carbon was added and stirred for 20 minutes. The mass was filtered and the bed washed with dichloromethane. The filtrate was distilled under vacuum, acetone (5 times by volume to the weight of crude) was added and cooled to 30° C. The reaction mass was maintained for 10 minutes at 30° C., filtered and washed with acetone. The solid was dried at about 58° C. to a constant weight to afford 11.2 g of the title compound.

### EXAMPLE 7

### Preparation of 3-[2-[4-(6-fluoro-1,2-benzisoxazol-3-yl)-1-piperidinyl]ethyl]-6,7,8,9-tetrahydro-9-hydroxy-2-methyl-4H-pyrido[1,2-a]pyrimidin-4-one (Formula I)

3-(2-Chloroethyl)-6,7,8,9-tetrahydro-9-hydroxy-2-methyl-4H-pyrido[1,2-a]pyrimidin-4-one of Formula V (28 g), 6-fluoro-3-(4-piperidinyl)-1,2-benzisoxazole mono hydrochloride of Formula VI (22.12 g), methanol (280 mL) and N-(1-methylethyl)-2-propanamine (23.23 g) were charged into a flask and stirred for about 16 hours at about 60° C. After completion of the reaction, the solvent was distilled completely under vacuum. The residue was dissolved in dichloromethane (280 mL) and washed with water (280 mL). The separated organic layer was dried over anhydrous sodium sulphate and the solvent was removed completely at about 50° C. under vacuum to afford a residue. Acetone (84 mL) was added to the residue and stirred for about 10-15 minutes at about 40° C. to about 50° C. The mixture was cooled to about 0° C. to about 5° C., stirred, filtered, washed with acetone (28 mL) and dried to afford the title compound (20.4 g).

### EXAMPLE 8

### Preparation of 3-[2-[4-(6-fluoro-1,2-benzisoxazol-3-yl)-1-piperidinyl]ethyl]-6,7,8,9-tetrahydro-9-hydroxy-2-methyl-4H-pyrido[1,2-a]pyrimidin-4-one (Formula I), Using Microwave Radiation

3-(2-Chloroethyl)-6,7,8,9-tetrahydro-9-hydroxy-2-methyl-4H-pyrido[1,2-a]pyrimidin-4-one (5 g), 6-fluoro-3-(4-piperidinyl)-1,2-benzisoxazole monohydrochloride (5.2 g), methanol (200 mL) and N-(1-methylethyl)-2-propanamine (4.16 g) were charged into a microwave reactor vessel. Microwave radiation was applied for 7 minutes (temperature raised to 70° C.). After completion of the reaction, solvent was distilled completely under vacuum. To the residue, water (25 mL) was added and the mixture was extracted with dichloromethane (3×25 mL). Combined organic layer was distilled under vacuum at 40° C. to afford a residue. To the residue, acetone (100 mL) was added and heated to reflux for 10 minutes. The mass cooled to 30° C., maintained for about 10-15 minutes, filtered and washed with acetone (5 mL). The solid was dried at 58° C. to afford 4.5 g of the title compound.

### EXAMPLE 9

### Purification of 3-[2-[4-(6-fluoro-1,2-benzisoxazol-3-yl)-1-piperidinyl]ethyl]-6,7,8,9-tetrahydro-9-hydroxy-2-methyl-4H-pyrido[1,2-a]pyrimidin-4-one

3-[2-[4-(6-Fluoro-1,2-benzisoxazol-3-yl)-1-piperidinyl]ethyl]-6,7,8,9-tetrahydro-9-hydroxy-2-methyl-4H-pyrido[1,2-a]pyrimidin-4-one (8.5 g) and dichloromethane (42.5 mL) were charged into a clean and dry round bottom flask. The mixture was heated to reflux and maintained for 15 minutes, then was cooled to 27° C. and stirred about 60 minutes. The obtained solid was filtered, washed with ethyl acetate (4 mL) and dried at 50° C. under vacuum to afford 4.6 g of the title compound.

The mother liquor was distilled under vacuum, ethyl acetate was added and then the mixture was stirred for 40 minutes. Filtered the solid, washed with ethyl acetate and dried at 50° C. under vacuum to afford 2.8 g of the second crop of the title compound.

### EXAMPLE 10

### Purification of 3-[2-[4-(6-fluoro-1,2-benzisoxazol-3-yl)-1-piperidinyl]ethyl]-6,7,8,9-tetrahydro-9-hydroxy-2-methyl-4H-pyrido[1,2-a]pyrimidin-4-one

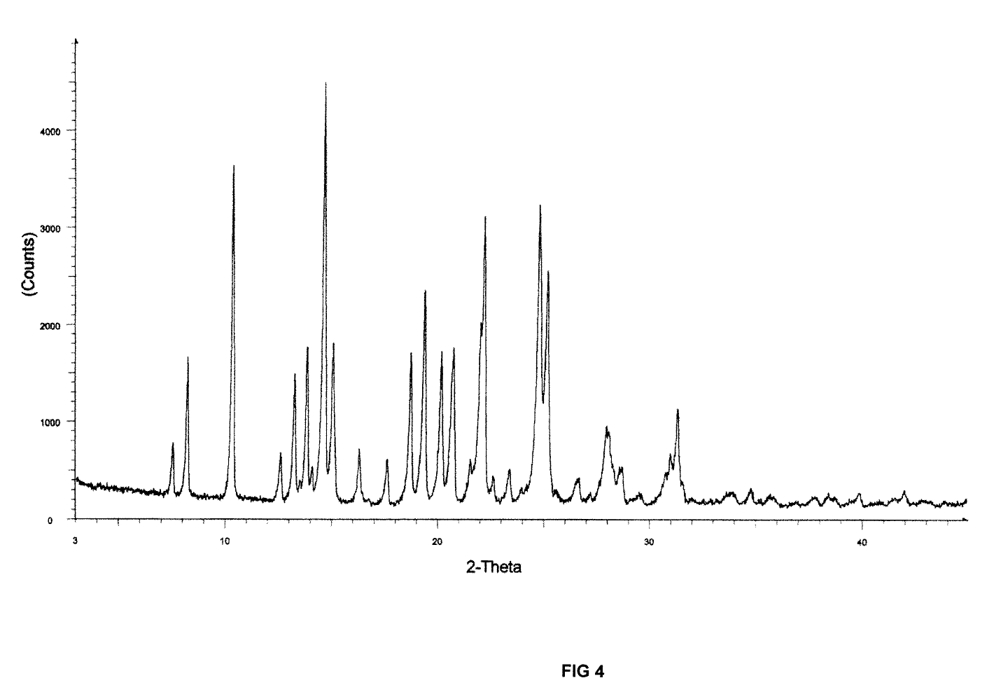

3-[2-[4-(6-Fluoro-1,2-benzisoxazol-3-yl)-1-piperidinyl]ethyl]-6,7,8,9-tetrahydro-9-hydroxy-2-methyl-4H-pyrido[1,2-a]pyrimidin-4-one (16 g) and toluene (240 mL) were placed into a flask. The mixture was heated to reflux and activated carbon (4.8 g) was charged into the mass and stirred about 30 minutes at about 90° C. The mass was filtered hot through a celite bed and the bed was washed with toluene (80 mL). The filtrate was cooled to about 25° C. to about 35° C. and stirred for about 60 minutes. The obtained solid was filtered, washed with toluene (80 mL) and dried at about 70° C. under vacuum. The above dry solid and toluene (120 mL) were charged into a flask. The mixture was heated to reflux for dissolution and activated carbon (2.4 g) was charged into the mass and stirred about 30 minutes at about 90° C. The mass was filtered hot through a celite bed and the bed was washed with toluene (40 mL). The filtrate was cooled to about 25° C. to about 35° C. and stirred for about 60 minutes. The obtained solid was filtered, washed with toluene (40 mL) and dried at 70° C. under vacuum. The dry solid and dichloromethane (16 mL) were charged into a flask and stirred for about 90 minutes at about 25° C. to about 35° C. The solid was filtered, washed with cyclohexane (8 mL) and dried at about 70° C. under vacuum to afford the title compound (3.9 g, HPLC purity 99.5%). FIG. 4 is an XRPD pattern of the crystalline paliperidone obtained.

### EXAMPLE 11

### Preparation of 3-(2-chloromethyl)-2-methyl-9-(phenylmethoxy)-4H-pyrido[1,2a]pyrimidine-4-one (Formula XI)

Phosphoryl chloride (210.8 g) was charged into a flask and cooled to about 5° C.-10° C. A solution of 2-amino-3-benzyloxy pyridine (50 g) and 2-acetyl butyrolactone (160 g) in toluene (250 mL) was added over a period of about 45 minutes at 5° C.-10° C., and then stirred for about 6 to about 8 hours at about 70° C. to about 80° C. After completion of the reaction, the reaction mass was poured into crushed ice followed by addition of water, stirred for about 10 to about 15 minutes, and layers were separated. The aqueous layer was washed with toluene (2×150 mL), the separated aqueous layer was cooled to about 10° C. to about 15° C. and the pH was adjusted to about 7 to about 8 with aqueous ammonia (400 mL) and stirred for about 30 to about 45 minutes at about 10° C. to about 15° C. The solid was filtered, washed with water (250 ml). Charged wet solid and water (500 ml) into RBF and stirred at 25-35° C. for 30-40 minutes. The solid was filtered and washed with water (250 ml) and dried at about 50° C. under vacuum. The solid (66 g) and isopropyl alcohol (330 mL) were charged into a flask and stirred for about 45 minutes at reflux and then cooled to about 25° C. to about 35° C. The solid was filtered, washed with isopropyl alcohol (132 mL) and dried at 50° C. under vacuum to afford the title compound (52 g).

1. A process for preparing paliperidone of Formula I, 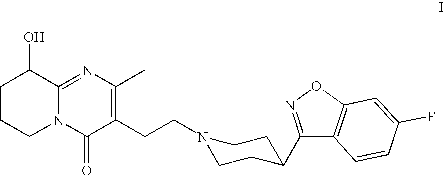
comprising:
a) reducing 3-(2-chloroethyl)-2-methyl-9-hydroxy-4H-pyrido[1,2-a]pyrimidin-4-one of Formula IV, 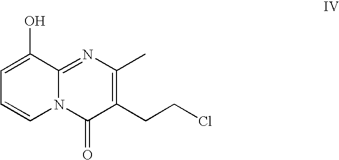
or 3-(2-chloromethyl)-2-methyl-9-(phenylmethoxy)-4H-pyrido[1,2a]pyrimidine-4-one of Formula XI, 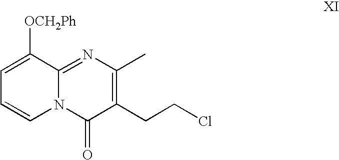
wherein Ph is a phenyl group, in the presence of acetic anhydride, a reducing agent, and optionally in the presence of a solvent, to produce 3-(2-chloroethyl)-6,7,8,9-tetrahydro-9-acetyloxy-2-methyl-4H-pyrido[1,2-a]pyrimidin-4-one of Formula VIII; 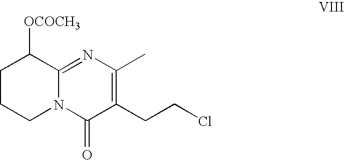
b) optionally, reacting 3-(2-chloroethyl)-6,7,8,9-tetrahydro-9-acetyloxy-2-methyl-4H-pyrido[1,2-a]pyrimidin-4-one of Formula VIII with a pharmaceutically acceptable acid to form a salt; and
c) reacting 3-(2-chloroethyl)-6,7,8,9-tetrahydro-9-acetyloxy-2-methyl-4H-pyrido[1,2-a]pyrimidin-4-one of Formula VIII or a salt thereof with 6-fluoro-3-(4-piperidinyl)-1,2-benzisoxazole of Formula II or a salt thereof, 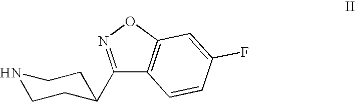
to produce paliperidone of Formula I.

2. The process of claim 1, wherein 3-(2-chloroethyl)-6,7,8,9-tetrahydro-9-acetyloxy-2-methyl-4H-pyrido[1,2-a]pyrimidin-4-one of Formula VIII is purified before further reaction.

3. The process of claim 1, wherein step c) comprises converting 3-(2-chloroethyl)-6,7,8,9-tetrahydro-9-acetyloxy-2-methyl-4H-pyrido[1,2-a]pyrimidin-4-one of Formula VIII into 3-(2-chloroethyl)-6,7,8,9-tetrahydro-9-hydroxy-2-methyl-4H-pyrido[1,2-a]pyrimidin-4-one of Formula III, or a salt thereof, 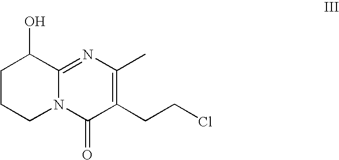
and reacting with 6-fluoro-3-(4-piperidinyl)-1,2-benzisoxazole of Formula II, or a salt thereof, to form paliperidone.

4. The process of claim 1, wherein a reducing agent in step a) comprises palladium on activated carbon.

5. The process of claim 1, wherein a solvent used in step a) comprises acetic acid.

6. The process of claim 1, wherein a base used in step b) comprises N-(1-methylethyl)-2-propanamine.

7. The process of claim 1, wherein step b) is carried out in the presence of microwave radiation.

8. Paliperidone prepared according to the process of claim 1 and having less than about 0.5 percent by weight of impurities, as determined by high performance liquid chromatography.

9. A pharmaceutical composition containing paliperidone prepared by the process of claim 1 and at least one pharmaceutically acceptable excipient.

10. A process for preparing 3-(2-chloroethyl)-6,7,8,9-tetrahydro-9-acetyloxy-2-methyl-4H-pyrido[1,2-a]pyrimidin-4-one of Formula VIII, 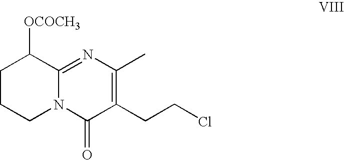
or a salt thereof, comprising:
a) reducing 3-(2-chloroethyl)-2-methyl-9-hydroxy-4H-pyrido[1,2-a]pyrimidin-4-one of Formula IV, 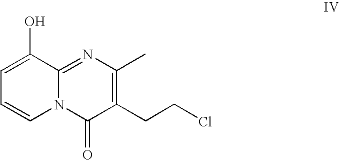
or 3-(2-Chloromethyl)-2-methyl-9-(phenylmethoxy)-4H-Pyrido[1,2a]pyrimidine-4-one of Formula XI, 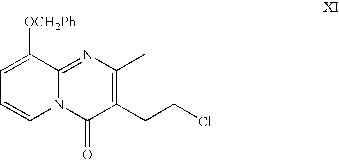
in the presence of acetic anhydride and a reducing agent, and optionally in the presence of a solvent; and
b) optionally, reacting the compound of Formula VIII with a pharmaceutically acceptable acid to form a salt.

11. The process of claim 10, wherein a reducing agent comprises platinum oxide, platinum on activated carbon, palladium on activated carbon, palladium on barium sulfate, palladium on calcium carbonate, palladium on barium carbonate, copper-chromium oxide, rhodium, cobalt, or ruthenium.

12. The process of claim 10, wherein a reducing agent comprises palladium on activated carbon.

13. The process of claim 10, wherein a solvent comprises a ketone, a halogenated hydrocarbon, a hydrocarbon, an ester, acetic acid, or a mixture of two or more thereof.

14. The process of claim 10, wherein a solvent is acetic acid.

15. A process for preparing paliperidone of Formula 1, comprising reacting 3-(2-chloroethyl)-6,7,8,9-tetrahydro-9-acetyloxy-2-methyl-4H-pyrido[1,2-a]pyrimidin-4-one of Formula VIII, 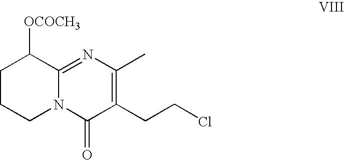
or a salt thereof, or 3-(2-chloroethyl)-6,7,8,9-tetrahydro-9-hydroxy-2-methyl-4H-pyrido[1,2-a]pyrimidin-4-one of Formula III, 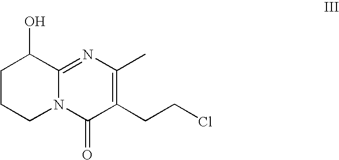
or a salt thereof, with 6-fluoro-3-(4-piperidinyl)-1,2-benzisoxazole of Formula II, 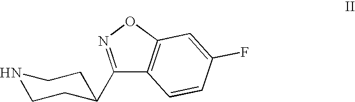
or a salt thereof, in the presence of microwave radiation.

16. The process of claim 15, wherein paliperidone is purified by recrystallizing or slurrying paliperidone in a solvent.

17. Paliperidone prepared according to the process of claim 15 and having less than about 0.5 percent by weight of impurities, as determined by high performance liquid chromatography.

18. Paliperidone prepared according to the process of claim 15 and having less than about 0.2 percent by weight of impurities, as determined by high performance liquid chromatography.

19. Paliperidone prepared according to the process of claim 15 and having less than about 0.1 percent by weight of impurities, as determined by high performance liquid chromatography.
